(19) United States
(12) Patent Application Publication
(10) Pub. No.: US 2024/0390574 A1
(43) Pub. Date: Nov. 28, 2024

(54) Electronic Device

(71) Applicant: TERUMO KABUSHIKI KAISHA, Tokyo (JP)

(72) Inventors: Shun DEMIZU, Hadano-shi (JP); Hideyuki SATO, Sendai-shi (JP)

(73) Assignee: TERUMO KABUSHIKI KAISHA, Tokyo (JP)

(21) Appl. No.: 18/795,913

(22) Filed: Aug. 6, 2024

Related U.S. Application Data
(63) Continuation of application No. PCT/JP2023/003852, filed on Feb. 6, 2023.

(30) Foreign Application Priority Data
Feb. 17, 2022 (JP) 2022-023244

(51) Int. Cl. A61M 5/142; A61J 1/16; A61M 5/14

(52) U.S. Cl. CPC A61M 5/14228; A61J 1/16; A61M 5/1413

(57) ABSTRACT

An electronic device according to the present disclosure is an electronic device on which a mounting body can be mounted so as to cover an opening communicating with inside in which an electronic component is stored, the electronic device including at least an eccentric side view in which, in a case in which a surface on which the opening is formed is set as an upper surface and a surface located on an opposite side of the upper surface is set as a lower surface, a lower end portion of the lower surface, which is located on a lowest side in a vertical direction, is located only on one side of a virtual centroidal line extending downward in the vertical direction from a center of gravity of the electronic device in any side view in a vertical posture in which the upper surface is directed upward in the vertical direction.

### CROSS-REFERENCE TO RELATED APPLICATIONS

This is a bypass continuation of PCT Application No. PCT/JP2023/003852, filed on Feb. 6, 2023, which claims priority to Japanese Patent Application No. 2022-023244, filed on Feb. 17, 2022. The entire contents of these applications are incorporated by reference herein.

### BACKGROUND

The present disclosure relates to an electronic device.

In related art, there has been known an electronic device on which a mounting body can be mounted so as to cover an opening communicating with inside in which an electronic component is stored. PCT Publication No. WO 2021/106425 A (“Patent Literature 1”) discloses this type of electronic device. A pump body as the electronic device of Patent Literature 1 is configured such that an infusion cartridge as the mounting body can be mounted. In addition, fingers that operate to act on the infusion cartridge side are disposed in the opening of the pump body described in Patent Literature 1. The infusion cartridge described in Patent Literature 1 is mounted on the pump body so as to cover the opening of the pump body.

### SUMMARY

The opening of the pump body as the electronic device described in Patent Literature 1 is exposed to outside in a state in which the infusion cartridge as the mounting body is removed. If liquid such as a medicinal solution enters inside of the pump body through the opening, there is a risk that a failure may occur in the electronic component stored in the pump body.

An object of the present disclosure is to provide an electronic device capable of avoiding entry of liquid into its inside through an opening even in a state in which a mounting body to be mounted so as to cover the opening is removed.

An electronic device according to a first aspect of the present disclosure is an electronic device on which a mounting body can be mounted so as to cover an opening communicating with inside in which an electronic component is stored, the electronic device including, in a case in which a surface on which the opening is formed is set as an upper surface, and a surface located on an opposite side of the upper surface is set as a lower surface, at least an eccentric side view in which a lower end portion located on a lowest side in a vertical direction of the lower surface is located on only one side of a virtual centroidal line extending downward in the vertical direction from a center of gravity of the electronic device in any side view in a vertical posture in which the upper surface is directed upward in the vertical direction.

As one embodiment of the present disclosure, in the eccentric side view, the lower surface includes the lower end portion and one or two inclined portions extending from the lower end portion so as to be inclined with respect to the vertical direction.

As one embodiment of the present disclosure, in the eccentric side view, the lower surface is formed with an arc surface formed with an arc line including the lower end portion and the one or two inclined portions, and the center of gravity is located above a position of a center of the arc surface in the vertical direction.

As one embodiment of the present disclosure, the electronic device includes a movable portion that is exposed from the opening of the upper surface and is movable so as to act on the mounting body in a mounted state, a drive unit that drives the movable portion, and a housing that covers a periphery of the electronic component and the drive unit and includes the lower surface.

As one embodiment of the present disclosure, in the eccentric side view, the electronic device includes a first side surface facing the one side across the virtual centroidal line and a second side surface facing the other side across the virtual centroidal line, and the electronic device includes a display unit exposed to the first side surface and capable of displaying device information.

An electronic device according to one embodiment of the present disclosure includes an operation unit that is exposed to the first side surface and allows input of information from outside.

According to the present disclosure, it is possible to provide an electronic device capable of avoiding entry of liquid into its inside through an opening even in a state in which a mounting body to be mounted so as to cover the opening is removed.

### BRIEF DESCRIPTION OF THE DRAWINGS

FIG. 1 is a view illustrating an electronic device as a first embodiment of an electronic device according to the present disclosure.

FIG. 2 is a front view of an infusion pump including a pump body as a second embodiment of the electronic device according to the present disclosure.

FIG. 3A is a view illustrating a state immediately before an infusion cartridge illustrated in FIG. 2 is mounted on the pump body illustrated in FIG. 2.

FIG. 3B is a view illustrating a state in which the infusion cartridge illustrated in FIG. 2 is being mounted on the pump body illustrated in FIG. 2.

FIG. 3C is a view illustrating a state in which mounting of the infusion cartridge illustrated in FIG. 2 on the pump body illustrated in FIG. 2 is completed.

FIG. 4 is a perspective view of the infusion cartridge alone illustrated in FIG. 2, and is a view illustrating a closed state in which a cover portion is closed with respect to a case portion.

FIG. 5 is a cross-sectional view of the infusion cartridge alone illustrated in FIG. 2, and is a view illustrating a state in which an infusion solution is not stored in a storage space of the infusion cartridge.

FIG. 6 is a cross-sectional view of the infusion cartridge alone illustrated in FIG. 2, and is a view illustrating a state in which the infusion solution is stored in the storage space of the infusion cartridge.

FIG. 7 is a perspective view of the pump body alone illustrated in FIG. 2.

FIG. 8 is a side view in a case in which the pump body illustrated in FIG. 7 is in a vertical posture, and is a view illustrating a side view other than an eccentric side view.

FIG. 9 is a side view in a case in which the pump body illustrated in FIG. 7 is in a vertical posture, and is a view illustrating an eccentric side view.

FIG. 10 is a cross-sectional view of the pump body taken along a line I-I in FIG. 8.

FIG. 11 is a view illustrating an example of a state in which liquid can drop from above the pump body illustrated in FIG. 7 in a vertical direction.

### DETAILED DESCRIPTION

Hereinafter, embodiments of an electronic device according to the present disclosure will be described by way of example with reference to the drawings. In the drawings, the same components are denoted by the same reference numerals.

### First Embodiment

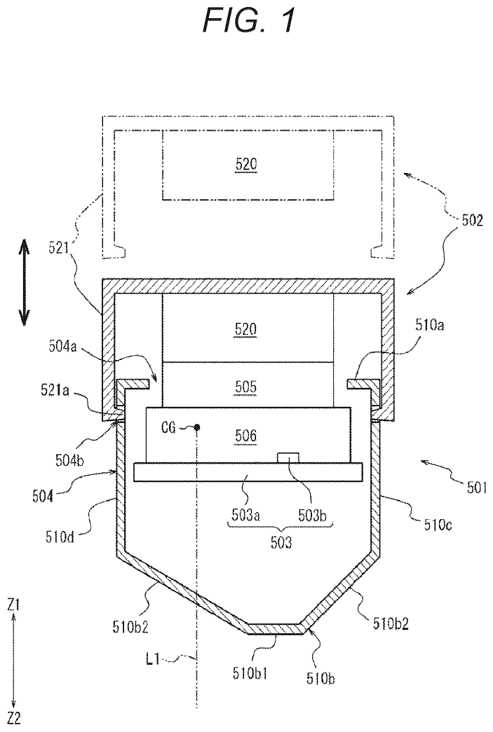

FIG. 1 is a view illustrating an electronic device 501 as one embodiment of an electronic device according to the present disclosure. As illustrated in FIG. 1, the electronic device 501 is configured such that a mounting body 502 can be mounted.

As illustrated in FIG. 1, the electronic device 501 stores an electronic component 503b therein. More specifically, the electronic device 501 of the present embodiment includes a circuit member 503. The circuit member 503 includes a plurality of electronic components 503b constituting an electronic circuit. Examples of the electronic components 503b include an integrated circuit mounted on a circuit board 503a.

An opening 504a communicating with inside in which the electronic component 503b is stored is formed on an outer surface of the electronic device 501. As illustrated in FIG. 1, the mounting body 502 can be mounted on the electronic device 501 so as to cover the opening 504a of the electronic device 501. FIG. 1 illustrates the mounting body 502 in a state of being mounted on the electronic device 501. In FIG. 1, the mounting body 502 in a state of being not mounted on the electronic device 501 is indicated by a two-dot chain line. As illustrated in FIG. 1, in the present embodiment, in a state in which the mounting body 502 is disposed so as to cover the opening 504a of the electronic device 501, a claw portion 521a of the mounting body 502 enters a recess or a through hole that is a claw receiving portion 504b of the electronic device 501. Accordingly, mounting of the mounting body 502 on the electronic device 501 is completed. However, a configuration for mounting the mounting body 502 on the electronic device 501 is not limited to the claw portion 521a and the claw receiving portion 504b of the present embodiment, and may be another configuration.

In addition, the mounting body 502 can be removed from the electronic device 501. If the mounting body 502 is removed from the electronic device 501, the opening 504a of the electronic device 501 is exposed to outside.

As illustrated in FIG. 1, the electronic device 501 of the present embodiment includes a housing 504 in which the opening 504a is formed. In addition, the electronic device 501 of the present embodiment includes a movable portion 505 that is exposed from the opening 504a and is movable so as to act on the mounting body 502 in the mounted state. Furthermore, the electronic device 501 of the present embodiment includes a drive unit 506 that drives the movable portion 505. A periphery of the circuit member 503 including the above-described electronic component 503b and the drive unit 506 is covered by the housing 504. In other words, the housing 504 stores the circuit member 503 and the drive unit 506 in a storage space inside.

In addition, the mounting body 502 of the present embodiment includes a cooperating portion 520 that implements a predetermined function in cooperation with the movable portion 505 of the electronic device 501, and a housing 521 that stores the cooperating portion 520. As illustrated in FIG. 1, in the mounting body 502 of the present embodiment, the housing 521 includes the above-described claw portion 521a to be locked by the claw receiving portion 504b of the electronic device 501.

As described above, in the electronic device 501 and the mounting body 502 of the present embodiment, in a mounted state in which the mounting body 502 is mounted on the electronic device 501, the electronic device 501 and the mounting body 502 cooperate to implement a predetermined function. More specifically, the electronic device 501 of the present embodiment is configured to implement a predetermined function in cooperation with the cooperating portion 520 of the mounting body 502 mounted on the electronic device 501 by the movable portion 505 exposed from the opening 504a being operated.

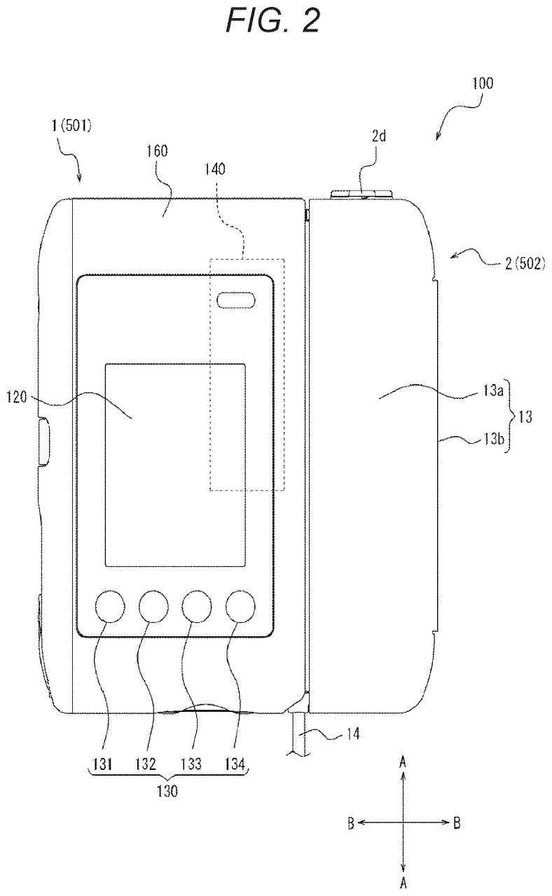

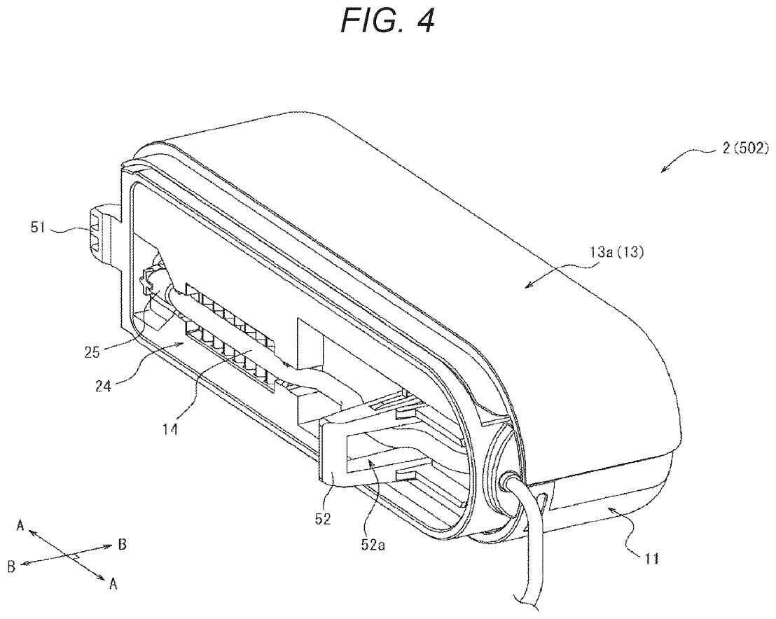

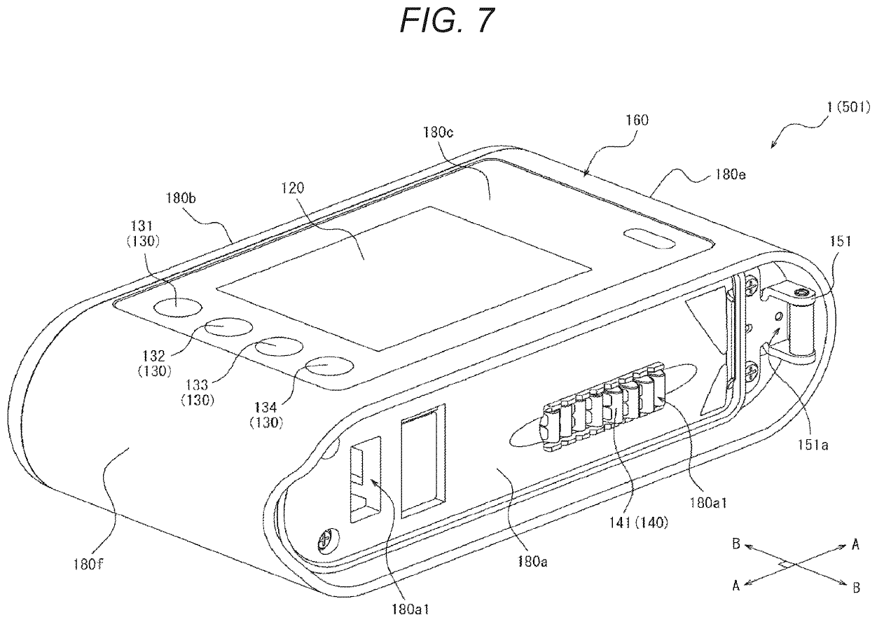

Examples of the electronic device 501 and the mounting body 502 having such a relationship include a pump body 1 and an infusion cartridge 2 of an infusion pump 100 to be described later (see FIGS. 2 to 11). Although details will be described later as a second embodiment, in a state in which the infusion cartridge 2 (see FIG. 2) that is the mounting body 502 is mounted on the pump body 1 (see FIG. 2) that is the electronic device 501, a plurality of fingers 141 (see FIG. 7, and the like) that are the movable portions 505 sequentially operable toward the infusion cartridge 2 side. The fingers 141 cooperate with a tube receiving portion 24 (see FIG. 4, and the like) that is the cooperating portion 520 of the mounting body 502 to draw a tube portion 14 (see FIG. 4, and the like) received by the tube receiving portion 24. As a result, a liquid delivery function of an infusion solution in the tube portion 14, which is one example of the predetermined function, is implemented.

However, the electronic device 501 only needs to have a configuration in which the mounting body 502 can be mounted so as to cover the opening 504a communicating with inside in which the electronic component 503b is stored, and a combination of the electronic device 501 and the mounting body 502 is not limited to the pump body and the infusion cartridge of the infusion pump described above.

Here, a surface of the electronic device 501 on which the opening 504a is formed will be referred to as an upper surface 510a, and a surface located on an opposite side of the upper surface 510a will be referred to as a lower surface 510b. As illustrated in FIG. 1, a posture in which the upper surface 510a of the electronic device 501 faces upward Z1 in the vertical direction will be referred to as a “vertical posture” of the electronic device 501. In such a case, at least an eccentric side view is included in any side view in which the electronic device 501 in the vertical posture is viewed from a horizontal direction (a direction orthogonal to the vertical direction in FIG. 1). The eccentric side view means a side view in which, among any side view of the electronic device 501 in the vertical posture, a lower end portion 510b1 located on the lowest side in the vertical direction of the lower surface 510b is located only on one side (the right side in FIG. 1) of a virtual centroidal line L1 extending downward Z2 in the vertical direction from a center of gravity CG of the electronic device 501. Here, as in the present embodiment, in a case in which the lower end portion 510b1 in the eccentric side view is a substantially horizontal plane formed with a line extending substantially horizontally, it means that the whole of the lower end portion 510b1 of the substantially horizontal plane is located only on one side (the right side in FIG. 1) of the virtual centroidal line L1. In the electronic device 501 illustrated in FIG. 1, the cross-sectional view of the electronic device 501 is illustrated for convenience of description, but a shape of the lower surface 510b illustrated in FIG. 1 is similar to a shape of the electronic device 501 in the eccentric side view.

As described above, as a result of the eccentric side view being included in any side view of the electronic device 501 in the vertical posture, if the lower surface 510b of the electronic device 501 in the vertical posture is placed on a horizontal placement surface, the electronic device 501 is likely to fall due to gravity. Specifically, the electronic device 501 illustrated in FIG. 1 is likely to fall toward the other side (left side in FIG. 1) opposite to one side (right side in FIG. 1) on which the lower end portion 510b1 is located, across the virtual centroidal line L1. Thus, the electronic device 501 of the present embodiment is less likely to be placed in a vertical posture where the upper surface 510a where the opening 504a is formed faces upward Z1 in the vertical direction. As a result, even if liquid unintentionally drips from above Z1 the electronic device 501 in the vertical direction, it is possible to prevent the liquid from directly dropping onto the upper surface 510a. Thus, even if there is liquid dropping from above Z1 the electronic device 501 in the vertical direction, the liquid is less likely to enter inside of the electronic device 501 through the opening 504a of the upper surface 510a. In other words, even in a state in which the mounting body 502 to be mounted so as to cover the opening 504a is removed from the electronic device 501, it is possible to prevent liquid from entering inside through the opening 504a. As a result, it is possible to prevent a failure of the electronic component 503b stored in the electronic device 501.

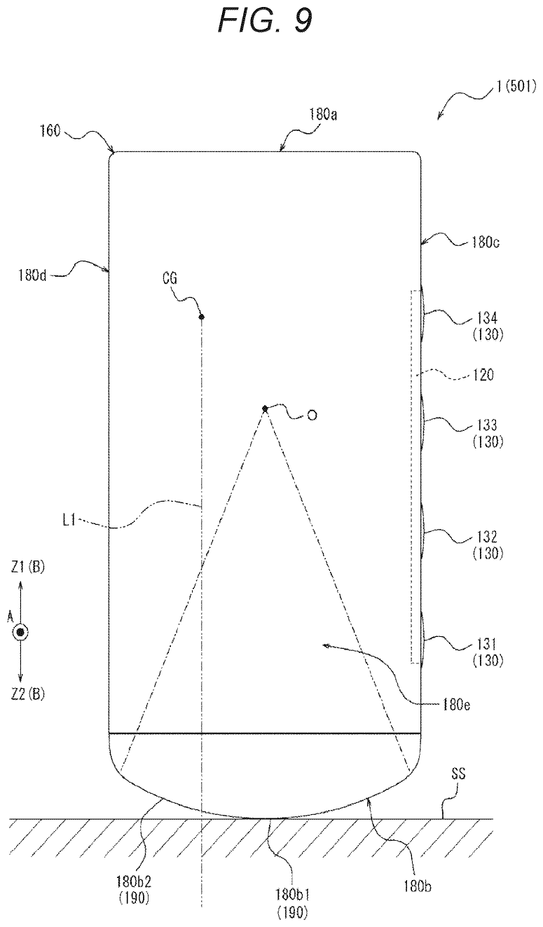

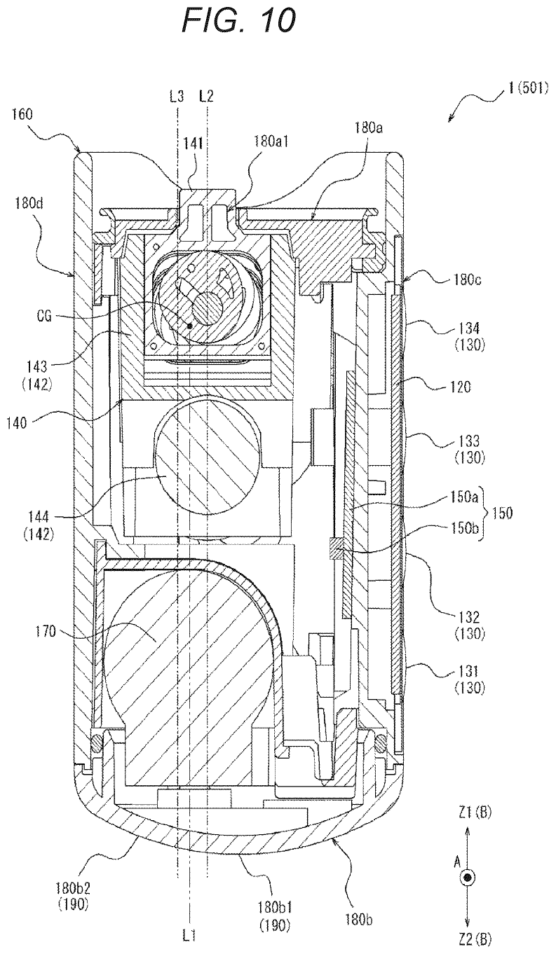

As illustrated in FIG. 1, the lower surface 510b of the electronic device 501 according to the present embodiment includes, but is not limited to, the lower end portion 510b1 having a substantially horizontal plane shape and formed with a line extending substantially horizontally, and two inclined portions 510b2 continuous to both ends of the lower end portion 510b1 in the eccentric side view. The lower end portion 510b1 may be, for example, a top portion constituted with approximately one point in the eccentric side view. In addition, the lower surface 510b may include, for example, only one inclined portion inclined with respect to the vertical direction in the eccentric side view. In such a case, the lower end portion 510b1 becomes one end of the inclined portion. Furthermore, the lower surface 510b may be, for example, an arc surface formed with a convex arc line in the eccentric side view. This will be described later in detail (see FIGS. 9 and 10).

In addition, as illustrated in FIG. 1, the electronic device 501 of the present embodiment includes a first side surface 510c facing one side across the virtual centroidal line L1 and a second side surface 510d facing the other side across the virtual centroidal line L1. The first side surface 510c and the second side surface 510d of the present embodiment are constituted with planes extending in the vertical direction and facing each other. Thus, the first side surface 510c and the second side surface 510d of the present embodiment extend in the vertical direction in the eccentric side view as in the cross-sectional view illustrated in FIG. 1. However, the first side surface 510c and the second side surface 510d are not limited to such planes extending in the vertical direction. The first side surface 510c and the second side surface 510d may be, for example, a concave curved surface, or the like.

### Second Embodiment

Next, another embodiment of the electronic device 501 according to the present disclosure will be described with reference to FIGS. 2 to 11. FIG. 2 is a front view of the infusion pump 100 including the pump body 1 as the electronic device 501 according to the present embodiment. As illustrated in FIG. 2, the infusion pump 100 includes the pump body 1 as the electronic device 501 and the infusion cartridge 2 as the mounting body 502. The infusion pump 100 illustrated in FIG. 2 can be used as, for example, a patient controlled analgesia (PCA) pump but is not particularly limited thereto. In the infusion pump 100 of the present embodiment, the pump body 1 is reusable by replacing the disposable infusion cartridge 2.

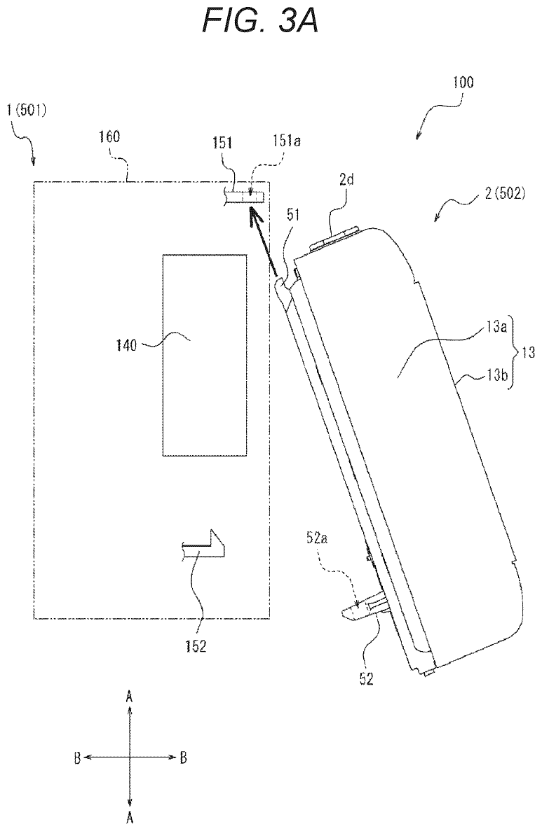

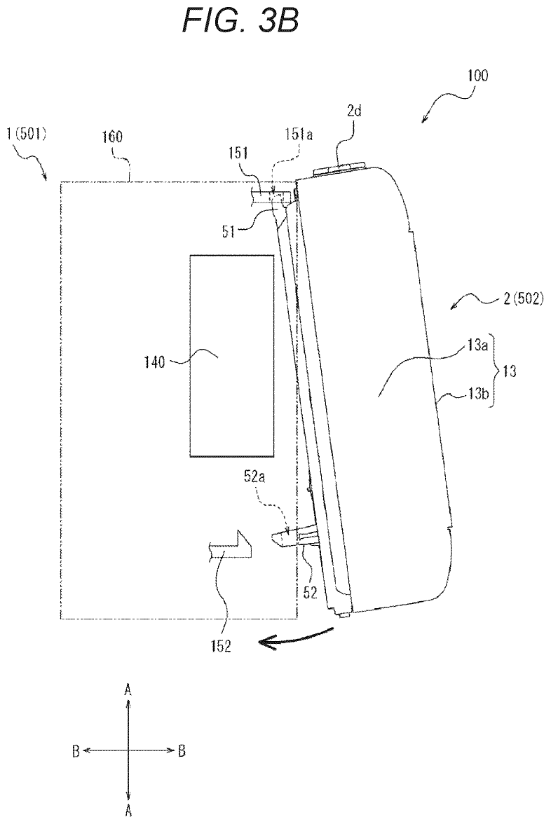

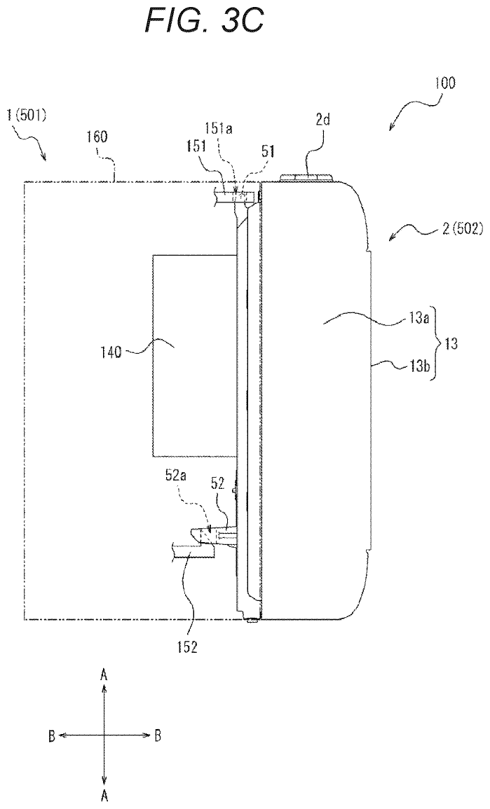

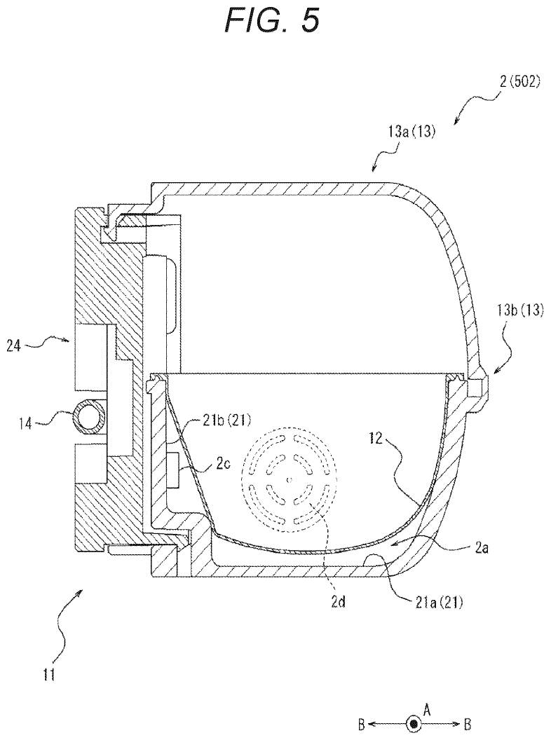

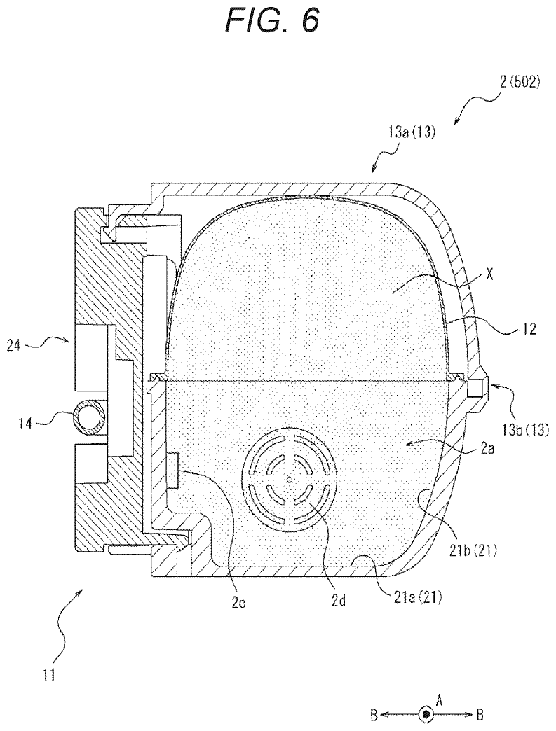

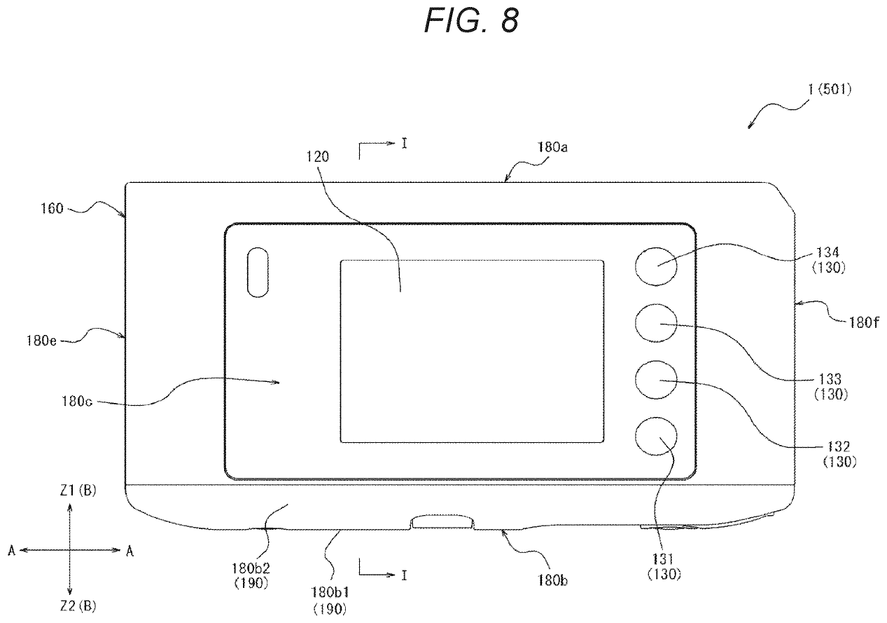

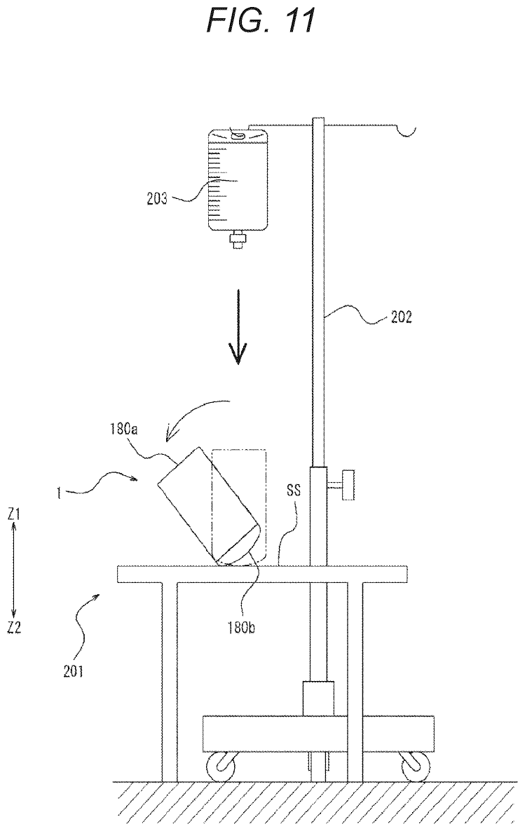

FIGS. 3A to 3C are explanatory diagrams illustrating operation when the infusion cartridge 2 is mounted on the pump body 1. FIG. 4 is a perspective view of the infusion cartridge 2 alone, and is a view illustrating a state in which a cover portion 13 is closed with respect to a case portion 11. FIGS. 5 and 6 are cross-sectional views of the infusion cartridge 2 alone. FIG. 5 illustrates a state in which the infusion solution is not stored in a storage space 2a of the infusion cartridge 2. FIG. 6 illustrates a state in which the infusion solution X is stored in the storage space 2a of the infusion cartridge 2. FIG. 7 is a perspective view of the pump body 1 alone. FIGS. 8 and 9 are side views of the pump body 1 in a vertical posture as viewed from different positions. In particular, FIG. 9 illustrates the eccentric side view of the pump body 1 in the vertical posture. FIG. 10 is a cross-sectional view of the pump body 1 taken along a line I-I in FIG. 8. FIG. 11 is a view illustrating an example of a state in which liquid can drop from above Z1 the pump body 1 in the vertical direction.

First, outline of the pump body 1 and the infusion cartridge 2 of the present embodiment will be described.

### [Pump Body 1]

As illustrated in FIG. 2, a display unit 120 on which various kinds of information are displayed and an operation unit 130 in which operation switches, and the like, are arranged are disposed on a front surface of the pump body 1. Various kinds of device information of the pump body 1 can be displayed on the display unit 120. Specifically, the display unit 120 displays, for example, a liquid delivery speed, a cumulative dose, and the like. Furthermore, the display unit 120 may be a liquid crystal screen with a touch panel for setting a liquid delivery speed, and the like. The operation unit 130 is configured to allow input of information from outside. The operation switches of the operation unit 130 illustrated in FIG. 2 include a fast delivery switch 131, a start switch 132, a stop switch 133, and a power switch 134. While the fast delivery switch 131 is depressed, liquid can be delivered at a liquid delivery speed higher than the set liquid delivery speed (mL/h). By the start switch 132 being depressed, liquid delivery can be started. By the stop switch 133 being depressed, the liquid delivery is forced to stop. By the power switch 134 being depressed, ON/OFF of a power supply of the pump body 1 can be switched. However, the operation unit 130 may include another operation switch, or may be constituted with another known means such as a touch panel. The display unit 120 and the operation unit 130 make it possible to deliver an infusion solution such as a medicinal solution into a living body of a patient, or the like, while controlling a delivery amount.

In addition, as illustrated in FIGS. 7, 10, and the like, the pump body 1 includes a liquid delivery unit 140 that sandwiches the tube portion 14 of the infusion cartridge 2 between the pump body 1 and the tube receiving portion 24 (see FIGS. 4 to 6) to be described later of the infusion cartridge 2 to be mounted and delivers the infusion solution in the tube portion 14 from a flow path upstream side to a flow path downstream side. As illustrated in FIGS. 7 and 10, the liquid delivery unit 140 of the present embodiment includes a plurality of fingers 141 as the movable portions 505 and a drive unit 142 (see FIG. 10) that drives the fingers 141. The plurality of fingers 141 are disposed on a facing surface 180a of the pump body 1 facing the tube receiving portion 24 (see FIGS. 3 and 4) to be described later located on a side surface of the infusion cartridge 2. The plurality of fingers 141 are arranged along an extending direction A of the tube portion 14 in a state in which the infusion cartridge 2 is mounted on the pump body 1. Each finger 141 is driven by the drive unit 142 so as to reciprocate in an opposite direction B with the tube receiving portion 24 (see FIGS. 4 to 6) to be described later of the infusion cartridge 2. As each finger 141 moves close to the infusion cartridge 2, the tube portion 14 is sandwiched between each finger 141 and the tube receiving portion 24. Accordingly, the tube portion 14 is compressed and closed. The drive unit 142 sequentially drives the fingers 141 from the flow path upstream side toward the flow path downstream side in the extending direction A of the tube portion 14. As illustrated in FIG. 10, the drive unit 142 includes, for example, a drive mechanism 143 that sequentially operates the plurality of fingers 141, and a drive source 144 such as an electric motor to be driven by a power source 170 such as a battery. As a result, the tube portion 14 is sequentially compressed and closed from the flow path upstream side toward the flow path downstream side and peristaltically moves. Thus, the infusion solution in the tube portion 14 can be delivered from the flow path upstream side toward the flow path downstream side.

As illustrated in FIG. 10, the pump body 1 stores the electronic component 150b therein. More specifically, the pump body 1 of the present embodiment includes a circuit member 150. The circuit member 150 includes a plurality of electronic components 150b constituting an electronic circuit. Examples of the electronic components 150b include an integrated circuit to be mounted on the circuit board 150a.

The pump body 1 of the present embodiment includes a housing 160. In other words, an outer surface of the pump body 1 of the present embodiment is covered with the housing 160. The display unit 120 and the operation unit 130 described above are exposed to outside from an opening formed in the housing 160. However, a gap between each of the display unit 120 and the operation unit 130 and the housing 160 is sealed by a seal member. In other words, the pump body 1 is configured such that liquid does not enter inside of the housing 160 from the opening of the housing 160 in which the display unit 120 and the operation unit 130 are disposed. The housing 160 may be formed with, for example, various resin materials.

In addition, as illustrated in FIGS. 7 and 10, an opening 180a1 covered by the infusion cartridge 2 in a state in which the infusion cartridge 2 is mounted on the pump body 1 is formed in the housing 160. The opening 180a1 is formed at a position of the facing surface 180a of the pump body 1. Thus, if the infusion cartridge 2 is removed from the pump body 1, the opening 180a1 of the pump body 1 is exposed to outside. In this event, the fingers 141 stored in the opening 180a1 are also exposed to outside from the opening 180a1.

The housing 160 of the pump body 1 of the present embodiment may be integrally formed with a single member or may be formed with two or more members.

The pump body 1 is not limited to the configuration of the present embodiment. The pump body 1 may include a portion different from the above-described portion, for example, a notification unit such as a bubble detection sensor unit or a blockage sensor unit. In addition, as described above, the liquid delivery unit 140 of the pump body 1 of the present embodiment has a configuration in which the tube portion 14 is pressed by the plurality of fingers 141, but may include the movable portion 505 different from the fingers 141 as long as the infusion solution in the tube portion 14 can be delivered.

As described above, the pump body 1 of the present embodiment includes the housing 160 including the facing surface 180a in which the opening 180a1 is formed. In addition, the pump body 1 of the present embodiment includes the fingers 141 as the movable portions 505 exposed from the opening 180a1 and movable to act on the infusion cartridge 2 in the mounted state. Furthermore, the pump body 1 of the present embodiment includes the drive unit 142 that drives the fingers 141 as the movable portions 505. A periphery of the circuit member 150 including the above-described electronic component 150b and the drive unit 142 is covered by the housing 160. In other words, the housing 504 stores the circuit member 150 and the drive unit 142 in a storage space inside.

In a state in which the infusion cartridge 2 is mounted on the pump body 1, the plurality of fingers 141 as the movable portions 505 exposed from the opening 180a1 are sequentially operable toward the infusion cartridge 2. Thereby, the plurality of fingers 141 cooperate with the tube receiving portion 24 of the infusion cartridge 2 to draw the tube portion 14 received in the tube receiving portion 24. As a result, it is possible to deliver the infusion solution such as a medicinal solution in the tube portion 14.

Here, the facing surface 180a of the pump body 1 where the opening 180a1 is formed will be hereinafter referred to as an “upper surface 180a” for convenience of description. A surface located on an opposite side of the upper surface 180a will be referred to as a lower surface 180b. Then, as illustrated in FIGS. 8 to 10, a posture in which the upper surface 180a of the pump body 1 faces upward Z1 in the vertical direction will be referred to as a “vertical posture” of the pump body 1. In such a case, at least an eccentric side view is included in any side view in which the pump body 1 in the vertical posture is viewed from the horizontal direction (a direction orthogonal to the vertical direction in FIGS. 8 to 10). In the pump body 1 of the present embodiment, the side view illustrated in FIG. 9 corresponds to the eccentric side view. The eccentric side view means a side view in which the lower end portion 180b1 located on the lowest side in the vertical direction of the lower surface 180b is located only on one side (the right side in FIG. 9) of the virtual centroidal line L1 extending from the center of gravity CG of the pump body 1 below Z2 in the vertical direction among any side view of the pump body 1. FIG. 10 illustrates a cross-sectional view of the pump body 1, but a shape of the lower surface 180b illustrated in FIG. 10 is similar to the shape in the eccentric side view in FIG. 9.

As described above, as a result of the eccentric side view being included in any side view of the pump body 1 in the vertical posture, if the lower surface 180b of the pump body 1 in the vertical posture is placed on a horizontal placement surface SS, the pump body 1 is likely to fall due to gravity. Specifically, in the pump body 1 in the eccentric side view illustrated in FIG. 9, the pump body 1 is likely to fall toward the other side (the left side in FIG. 9) opposite to one side (the right side in FIG. 9) where the lower end portion 180b1 is located, across the virtual centroidal line L1. Thus, the pump body 1 of the present embodiment is less likely to be placed in the vertical posture in which the upper surface 180a in which the opening portion 180a1 is formed faces upward Z1 in the vertical direction. As a result, even if liquid unintentionally drips from above Z1 the pump body 1 in the vertical direction, it is possible to prevent the liquid from directly dropping onto the upper surface 180a. Thus, even if there is liquid dropping from above Z1 the pump body 1 in the vertical direction, the liquid is less likely to enter inside of the pump body 1 through the opening 180a1 of the upper surface 180a. In other words, even in a state in which the infusion cartridge 2 to be mounted so as to cover the opening 180a1 is removed from the pump body 1, it is possible to prevent the liquid from entering inside through the opening 180a1. As a result, it is possible to prevent a failure of the electronic component 150b stored in the pump body 1.

As illustrated in FIG. 11, the pump body 1 may be placed on the placement surface SS of a placement table 201 located below Z2 in the vertical direction of an infusion bag 203 suspended on a stand 202. In such a case, liquid such as an infusion solution stored in the infusion bag 203 may be dripped onto the pump body 1. However, as described above, the eccentric side view is included in any side view of the pump body 1 in the vertical posture, and thus, the pump body 1 in the vertical posture is likely to fall. As a result, even if there is liquid dripped from the infusion bag 203, the liquid is less likely to directly drop onto the upper surface 180a, and it is possible to prevent the liquid from entering inside through the opening 180a1 of the upper surface 180a.

Further details of the configuration of the pump body 1 of the present embodiment in the eccentric side view will be described later.

### [Infusion Cartridge 2]

The infusion cartridge 2 can be mounted on the pump body 1. First, operation of mounting the infusion cartridge 2 on the pump body 1 will be described with reference to FIGS. 3A to 3C. FIG. 3A illustrates a state immediately before the infusion cartridge 2 is mounted on the pump body 1. FIG. 3B illustrates a state in which the infusion cartridge 2 is being mounted on the pump body 1. FIG. 3C illustrates a mounted state in which mounting of the infusion cartridge 2 on the pump body 1 is completed. As illustrated in FIGS. 3A to 3C, the pump body 1 of the present embodiment includes a receiving portion 151 in which a through hole 151a is formed, and a locking claw portion 152. In addition, the infusion cartridge 2 of the present embodiment includes a swing shaft portion 51 and a claw receiving portion 52 in which a through hole 52a is formed.

As illustrated in FIG. 3A, when the infusion cartridge 2 is mounted on the pump body 1, the swing shaft portion 51 of the infusion cartridge 2 is fitted into the through hole 151a of the receiving portion 151 of the pump body 1. Next, as illustrated in FIG. 3B, the infusion cartridge 2 is swung around the swing shaft portion 51 in a state in which the swing shaft portion 51 is fitted in the through hole 151a. As a result, as illustrated in FIG. 3C, the locking claw portion 152 of the pump body 1 enters the through hole 52a of the claw receiving portion 52 of the infusion cartridge 2 and locks the claw receiving portion 52. As a result, mounting of the infusion cartridge 2 on the pump body 1 is completed. More specifically, in the present embodiment, mounting of the infusion cartridge 2 on the pump body 1 is completed when the swing shaft portion 51 of the infusion cartridge 2 is fitted into the through hole 151a of the receiving portion 151 of the pump body 1, and the locking claw portion 152 of the pump body 1 enters the through hole 52a of the claw receiving portion 52 of the infusion cartridge 2. In this event, the infusion cartridge 2 is mounted on the pump body 1 so as to cover the opening 180a1 (see FIG. 7) of the upper surface 180a of the pump body 1.

In addition, when the infusion cartridge 2 mounted on the pump body 1 is removed from the pump body 1, it is only necessary to execute operation opposite to the mounting operation described above. First, the locking claw portion 152 of the pump body 1 is removed from the through hole 52a of the claw receiving portion 52 of the infusion cartridge 2. Next, the infusion cartridge 2 is swung in a direction opposite to an arrow direction in FIG. 3B. Next, the swing shaft portion 51 of the infusion cartridge 2 is removed from the through hole 151a of the receiving portion 151 of the pump body 1. As a result, removal of the infusion cartridge 2 from the pump body 1 is completed.

In the present embodiment, mounting/removal of the pump body 1 on/from the infusion cartridge 2 is implemented by the receiving portion 151 and the locking claw portion 152 of the pump body 1 and the swing shaft portion 51 and the claw receiving portion 52 of the infusion cartridge 2, but a configuration for implementing mounting/removal of the pump body 1 on/from the infusion cartridge 2 is not particularly limited. Thus, the pump body 1 and the infusion cartridge 2 may be detachable from each other using a configuration different from the present embodiment.

Next, details of the infusion cartridge 2 will be described with reference to FIGS. 4 to 6. As illustrated in FIGS. 5 and 6, the infusion cartridge 2 defines the storage space 2a capable of storing an infusion solution X. A filling port 2c and a ventilation port 2d are formed on an inner wall defining the storage space 2a. The storage space 2a can be filled with the infusion solution X from outside through the filling port 2c. A gas in the storage space 2a can be discharged to outside through the ventilation port 2d.

More specifically, the infusion cartridge 2 includes a case portion 11, a film portion 12, and a cover portion 13.

As illustrated in FIGS. 5 and 6, a recess 21 is formed in the case portion 11. The recess 21 of the present embodiment includes a planar bottom surface 21a and a side surface 21b continuously rising from an outer edge portion of the bottom surface 21a. An open end of the recess 21 is constituted with an end portion of the side surface 21b opposite to the bottom surface 21a side, that is, an edge portion of the recess 21. The recess 21 of the present embodiment has the above-described shape, but the shape of the recess 21 is not particularly limited. Thus, the bottom surface 21a of the recess 21 may be, for example, a concave curved surface. Further, the side surface 21b of the recess 21 may have a planar shape or a curved shape.

The film portion 12 has flexibility. As illustrated in FIGS. 5 and 6, the film portion 12 covers the open end of the recess 21 of the case portion 11 and defines the storage space 2a with the recess 21. The film portion 12 of the present embodiment is joined to the edge portion of the recess 21. The case portion 11 and the film portion 12 can be joined to each other by, for example, welding or the like, but a joining method is not particularly limited.

The case portion 11 has a fixed shape that is not deformed even by an internal pressure of the infusion solution X stored in the storage space 2a. As illustrated in FIGS. 5 and 6, the case portion 11 of the present embodiment includes a box body in which the recess 21 is formed and a plate body attached to a side surface side of the box body and in which the tube receiving portion 24 is formed, but is not limited to this configuration. The case portion 11 may be formed with, for example, a single member in which the box body and the plate body described above are integrated, or may be formed with three or more members.

The film portion 12 is deformed by the internal pressure of the infusion solution X stored in the storage space 2a. A thickness of the film portion 12 is smaller than a thickness of the case portion 11 described above. A volume of the storage space 2a varies due to deformation of the film portion 12. The storage space 2a of the present embodiment is defined between the case portion 11 and the film portion 12, but is not limited to this configuration. For example, the storage space 2a may be defined inside only by the bag-shaped film portion 12. In such a case, the case portion 11 may be a case body that covers the bag-shaped film portion 12. In addition, the case portion 11 may be separable from the bag-shaped film portion 12.

In this manner, the storage space 2a of the infusion cartridge 2 is defined by the recess 21 of the case portion 11 and the film portion 12 covering the recess 21. As described above, the filling port 2c and the ventilation port 2d are formed on an inner wall defining the storage space 2a.

The cover portion 13 is configured to be able to be open and closed with respect to the case portion 11. As illustrated in FIGS. 5 and 6, the cover portion 13 includes a cover body portion 13a and a hinge portion 13b.

The hinge portion 13b is connected to the case portion 11. The cover body portion 13a is rotatable around the hinge portion 13b between a closed state (see FIGS. 4 to 6) in which the cover body portion 13a covers the film portion 12 and an open state in which the cover body portion 13a does not cover the film portion 12.

The cover body portion 13a and the hinge portion 13b of the present embodiment are formed integrally with the case portion 11, but the present invention is not limited to this configuration. In other words, the cover portion 13 may be formed separately from the case portion 11 and may be rotatably attached to the case portion 11.

When the storage space 2a is filled with the infusion solution X, the cover body portion 13a is in an open state in which the cover body portion 13a does not cover the film portion 12. Thus, when the storage space 2a is filled with the infusion solution X, even if air bubbles enter the storage space 2a, the film portion 12 can be pushed in from the outside and easily deformed. Thus, when the storage space 2a is filled with the infusion solution X, the air bubbles in the storage space 2a can be easily moved.

In addition, after the storage space 2a is filled with the infusion solution X, the cover body portion 13a is in a closed state in which the cover body portion 13a covers the film portion 12. Then, the infusion cartridge 2 is mounted on the pump body 1 in a form in which the cover body portion 13a is in the closed state (see FIGS. 3A to 3C).

The tube portion 14 is connected to the case portion 11 so as to be able to fill the storage space 2a with the infusion solution X through the filling port 2c. As illustrated in FIG. 4, one end side of the tube portion 14 of the present embodiment communicates with the storage space 2a via an L-shaped connection tube portion 25. By connecting, for example, a syringe, or the like, to the other end side of the tube portion 14, the storage space 2a can be filled with the infusion solution X through the tube portion 14.

When the infusion pump 100 is used, the tube portion 14 is used as part of an infusion tube for administering the infusion solution X that fills the storage space 2a into the living body. In other words, the infusion pump 100 illustrated in FIG. 2 can deliver the infusion solution X discharged from the storage space 2a to the tube portion 14 through the filling port 2c to the flow path downstream side by causing peristaltic movement in the tube portion 14. On the flow path downstream side of the tube portion 14, for example, there is an indwelling needle indwelled in the living body, and the infusion solution X can be administered into the living body through the indwelling needle.

### [Configuration of Pump Body 1 in Eccentric Side View]

Next, a configuration of the pump body 1 in the eccentric side view will be described in detail. As described above, FIG. 9 is a side view illustrating the eccentric side view of the pump body 1. As described above, as a result of the eccentric side view being included in any side view of the pump body 1 in the vertical posture, if the lower surface 180b of the pump body 1 in the vertical posture is placed on the horizontal placement surface SS, the pump body 1 is likely to fall due to gravity. Thus, even if there is liquid dropping from above Z1 the pump body 1 in the vertical direction, the liquid is less likely to drop directly onto the upper surface 180a, and it is possible to prevent the liquid from entering inside of the pump body 1 from the opening 180al. As a result, it is possible to prevent a failure of the electronic component 150b stored in the pump body 1.

As illustrated in FIG. 9, in the eccentric side view of the pump body 1, the lower surface 180b of the present embodiment includes the lower end portion 180b1 and one or two (two in the present embodiment) inclined portions 180b2. The inclined portion 180b2 is a surface extending from the lower end portion 180b1 so as to be inclined with respect to the vertical direction. In the present embodiment, two inclined portions 180b2 continuous from both ends of the lower end portion 180b1 are provided in the eccentric side view (see FIG. 9) of the pump body 1. As described above, in the eccentric side view of the pump body 1, the lower surface 180b includes only the lower end portion 180b1 and the one or two inclined portions 180b2, and thus, when the pump body 1 in the vertical posture is placed on the placement surface SS, the lower surface 180b does not have a portion (for example, a protruding portion protruding below Z2 in the vertical direction from the inclined portion 180b2) that prevents the pump body 1 from falling during falling. Thus, the pump body 1 in the vertical posture is more likely to fall on the placement surface SS.

Further, as illustrated in FIG. 9, in the eccentric side view of the pump body 1, the lower surface 180b is formed with an arc surface 190 formed with an arc line including the lower end portion 180b1 and the one or two (two in the present embodiment) inclined portions 180b2. In the eccentric side view of the pump body 1, the center of gravity CG of the pump body 1 is located above Z1 a position of the center O of the arc surface 190 in the vertical direction. In this manner, the pump body 1 in the vertical posture is more likely to fall smoothly on the arc surface 190 on the placement surface SS. In the eccentric side view of the pump body 1, the center of gravity CG of the pump body 1 is located above Z1 the position of the center O of the arc surface 190 in the vertical direction, so that the pump body 1 in the vertical posture placed on the placement surface SS can be prevented from always returning to the vertical posture like a rising spill.

In addition, the pump body 1 of the present embodiment includes, in the eccentric side view (see FIG. 9), a first side surface 180c facing one side (the right side in FIG. 9) on which the lower end portion 180b1 is located across the virtual centroidal line L1, and a second side surface 180d facing the other side (the left side in FIG. 9) across the virtual centroidal line L1. The display unit 120 of the pump body 1 of the present embodiment is exposed to the first side surface 180c. In other words, the first side surface 180c of the present embodiment is a front surface of the pump body 1, and the display unit 120 is exposed on the front surface. With such a configuration, when the pump body 1 in the vertical posture falls on the placement surface SS, the pump body 1 falls so that the display unit 120 faces upward Z1 in the vertical direction. It is therefore possible to prevent the display unit 120 from being damaged by falling of the pump body 1.

Furthermore, in the present embodiment, not only the display unit 120 of the pump body 1 but also the operation unit 130 of the pump body 1 is exposed to the first side surface 180c. With such a configuration, it is possible to prevent the operation unit 130 from being damaged by falling of the pump body 1, prevent unintended operation of the operation unit, and prevent a malfunction. Further, the pump body 1 after falling on the placement surface SS can be easily operated by the operation unit 130.

A state in which the pump body 1 has fallen on the placement surface SS such that the first side surface 180c faces upward Z1 in the vertical direction will be referred to as a “fallen state”. In such a case, in the pump body 1 in the fallen state, the electronic component 150b is preferably disposed so as to be positioned above Z1 the opening 180a1 of the upper surface 180a in the vertical direction. In this way, even if liquid enters inside of the pump body 1 from the opening 180a1 in the fallen state, the liquid can be prevented from adhering to the electronic component 150b. In the present embodiment, in the pump body 1 in the fallen state, the circuit member 150 including the integrated circuit as the electronic component 150b is disposed so as to be positioned above Z1 in the vertical direction of the opening 180a1 of the upper surface 180a. Further, as illustrated in FIG. 10, the housing 160 preferably protrudes from the upper surface 180a. In FIG. 10, the first side surface 180c and the second side surface 180d of the pump body 1 constituted with the housing 160 protrude above Z1 in the vertical direction from the upper surface 180a. With such a configuration, in the pump body 1 in the fallen state, even if the liquid drops onto the first side surface 180c from above Z1 in the vertical direction, it is possible to prevent the liquid from reaching the upper surface 180a along the first side surface 180c.

The first side surface 180c and the second side surface 180d of the present embodiment are vertical planes extending along the vertical direction in the eccentric side view (see FIG. 9) and facing each other, but are not limited to this configuration. The first side surface 180c and the second side surface 180d may be, for example, concave curved surfaces. The first side surface 180c and the second side surface 180d may be, for example, convex curved surfaces. However, in a case in which the second side surface 180d is made a convex curved surface, a radius of curvature of the convex curved surface of the second side surface 180d is preferably larger than a radius of curvature of the arc surface 190 of the lower surface 180b in the eccentric side view (see FIG. 9). In this way, the pump body 1 after falling on the placement surface SS can be prevented from swinging by the second side surface 180d that is a convex curved surface, so that it is possible to improve position stability of the pump body 1 after falling.

The pump body 1 of the present embodiment includes a third side surface 180e and a fourth side surface 180f in addition to the upper surface 180a, the lower surface 180b, the first side surface 180c, and the second side surface 180d described above. The third side surface 180e is a side surface connecting one ends of the first side surface 180c and the second side surface 180d. The fourth side surface 180f is a side surface connecting the other ends of the first side surface 180c and the second side surface 180d. The upper surface 180a, the lower surface 180b, the first side surface 180c, the second side surface 180d, the third side surface 180e, and the fourth side surface 180f of the present embodiment are all constituted with the housing 160, but are not limited to this configuration. In other words, the outer surface of the pump body 1 may be constituted with the housing 160 or may be constituted with a member other than the housing 160.

Furthermore, as illustrated in FIG. 10, in the present embodiment, the center of gravity CG of the pump body 1 in the eccentric side view (see FIG. 9) is at a position sandwiched between a virtual line L2 passing through the center of gravity of the liquid delivery unit 140 and extending in the vertical direction and a virtual line L3 passing through the center of gravity of the power source 170 and extending in the vertical direction.

The electronic device according to the present disclosure is not limited to the specific configurations illustrated in the above-described embodiments, and various changes, modifications, and combinations can be made without departing from the scope of the claims.

### INDUSTRIAL APPLICABILITY

The present disclosure relates to an electronic device.

### REFERENCE SIGNS LIST

1 Pump body (example of electronic device)
2 Infusion cartridge (example of mounting body)
2a Storage space
2c Filling port
2d Ventilation port
11 Case portion
12 Film portion
13 Cover portion
13a Cover body portion
13b Hinge portion
14 Tube portion
21 Recess
21a Bottom surface
21b Side surface
24 Tube receiving portion
25 Connection tube portion
51 Swing shaft portion
52 Claw receiving portion
52a Through hole
100 Infusion pump
120 Display unit
130 Operation unit
131 Fast delivery switch
132 Start switch
133 Stop switch
134 Power switch
140 Liquid delivery unit
141 Finger (example of movable portion)
142 Drive unit
143 Drive mechanism
144 Drive source
150 Circuit member
150a Circuit board
150b Electronic component
151 Receiving portion
151a Through hole
152 Locking claw portion
160 Housing
170 Power source
180a Facing surface (upper surface)
180a1 Opening
180b Lower surface
180b1 Lower end portion
180b2 Inclined portion
180c First side surface
180d Second side surface
180e Third side surface
180f Fourth side surface
190 Arc surface
201 Placement table
202 Stand
203 Infusion bag
501 Electronic device
502 Mounting body
503 Circuit member
503a Circuit board
503b Electronic component
504 Housing
504a Opening
504b Claw receiving portion
505 Movable portion
506 Drive unit
510a Upper surface
510b Lower surface
510b1 Lower end portion
510b2 Inclined portion
510c First side surface
510d Second side surface
520 Cooperating portion
521 Housing
521a Claw portion
A Extending direction of tube portion
B Opposite direction
Z1 Above in vertical direction
Z2 Below in vertical direction
CG Center of gravity of electronic device
L1 Virtual centroidal line
L2 Virtual line passing through center of gravity of liquid delivery unit and extending in vertical direction

L3 Virtual line passing through center of gravity of power source and extending in vertical direction
O Center of arc surface in eccentric side view
SS Placement surface
X Infusion Solution

1. An electronic device on which a mounting body is mountable so as to cover an opening communicating with an inside of the electronic device in which an electronic component is stored, the electronic device comprising:
a case having an upper surface in which the opening is formed, and a lower surface opposite the upper surface; wherein:
in a side view in which the upper surface is directed upward in the vertical direction, a lower end portion located on a lowest side in a vertical direction of the lower surface is located on only one side of a virtual centroidal line extending downward in the vertical direction from a center of gravity of the electronic device.

2. The electronic device according to claim 1, wherein:
in the side view, the lower surface comprises:
the lower end portion, and
one or two inclined portions extending from the lower end portion so as to be inclined with respect to the vertical direction.

3. The electronic device according to claim 2, wherein:
in the side view:
the lower surface is formed with an arc surface formed with an arc line including the lower end portion and the one or two inclined portions, and
the center of gravity is located above a position of a center of the arc surface in the vertical direction.

4. The electronic device according to claim 1, comprising:
a movable portion that is exposed from the opening of the upper surface and is movable so as to act on the mounting body in a mounted state;
a drive unit configured to drive the movable portion; and
a housing that covers a periphery of the electronic component and the drive unit and includes the lower surface.

5. The electronic device according to claim 1, wherein:
in the side view, a first side surface faces a first direction with respect to the virtual centroidal line, and a second side surface facing a second direction with respect to the virtual centroidal line; and
the electronic device comprises a display unit exposed at the first side surface and configured to display device information.

6. The electronic device according to claim 1, comprising an operation unit that is exposed at the first side surface and is configured to recite input of information.

7. An infusion pump on which an infusion cartridge is mountable so as to cover an opening communicating with an inside of the infusion pump in which an electronic component is stored, the infusion pump comprising:
a case having an upper surface in which the opening is formed, and a lower surface opposite the upper surface;
wherein:
in a side view in which the upper surface is directed upward in the vertical direction, a lower end portion located on a lowest side in a vertical direction of the lower surface is located on only one side of a virtual centroidal line extending downward in the vertical direction from a center of gravity of the infusion pump.

8. The infusion pump according to claim 7, wherein:
in the side view, the lower surface comprises:
the lower end portion, and
one or two inclined portions extending from the lower end portion so as to be inclined with respect to the vertical direction.

9. The infusion pump according to claim 8, wherein:
in the side view:
the lower surface is formed with an arc surface formed with an arc line including the lower end portion and the one or two inclined portions, and
the center of gravity is located above a position of a center of the arc surface in the vertical direction.

10. The infusion pump according to claim 7, comprising:
a plurality of fingers that are exposed from the opening of the upper surface and are movable so as to act on the infusion cartridge in a mounted state;
a drive unit configured to drive the plurality of fingers; and
a housing that covers a periphery of the electronic component and the drive unit and includes the lower surface.

11. The infusion pump according to claim 7, wherein:
in the side view, a first side surface faces a first direction with respect to the virtual centroidal line, and a second side surface facing a second direction with respect to the virtual centroidal line; and
the infusion pump comprises a display unit exposed at the first side surface and configured to display device information.

12. The infusion pump according to claim 7, comprising an operation unit that is exposed at the first side surface and is configured to recite input of information.
